(19) United States
(12) Patent Application Publication
(10) Pub. No.: US 2020/0246244 A1
(43) Pub. Date: Aug. 6, 2020

(54) MOUSSE COMPOSITION

(71) Applicant: L'ORÉAL, Paris (FR)

(72) Inventor: Phi Hong DOAN, Clark, NJ (US)

(73) Assignee: L'ORÉAL, Paris (FR)

(21) Appl. No.: 16/263,464

(22) Filed: Jan. 31, 2019

(51) Int. Cl. A61K 8/81; A61K 8/34; A61K 8/20; A61K 8/29; A61K 8/04; A61Q 1/02

(52) U.S. Cl. CPC A61K 8/8158; A61K 8/34; A61K 8/20; A61K 8/29; A61K 2800/612; A61Q 1/02; A61K 2800/54; A61K 2800/33; A61K 2800/43; A61K 8/04

(57) ABSTRACT

The present invention relates to a mousse composition including water, at least one C2-C5 alcohol, at least one salt, at least one amphiphilic polymer, and at least one hydrophobic surface-treated pigment, as well as to methods for making-up and enhancing the appearance of keratinous materials using the mousse composition.

### FIELD OF THE INVENTION

The present invention relates to a mousse composition comprising water, at least one C2-C5 alcohol, at least one salt, at least one amphiphilic polymer, and at least one hydrophobic surface-treated pigment, as well as to methods for making-up and enhancing the appearance of keratinous materials using the mousse composition and methods for making the mousse composition.

### DISCUSSION OF THE BACKGROUND

U.S. Pat. No. 3,131,152 discloses aerosol foams including alcohol, water, surfactant and propellant.

U.S. Pat. No. 4,627,973 discloses aerosol mousses with a mixture of acetylated lanolin alcohol, an alkoxylated methyl glucose derivative and an alkoxylated lanolin derivative.

U.S. Pat. No. 5,167,950A discloses a high alcohol content aerosol mousse including a hydrocarbon propellant.

WO 1999/013827 discloses non-aerosol mousse compositions.

U.S. Pat. No. 6,264,964 discloses oil-in-water emulsion mousses incorporating crosslinked non-emulsifying siloxane elastomers.

EP 1086683 discloses a mousse composition containing oils.

U.S. Pat. No. 6,328,950 discloses pigment-containing foamable gels.

U.S. Pat. No. 5,487,884 discloses a mousse with propellants.

Although mousse compositions containing propellant are known, there remains a need for improved ways to prepare such compositions and for improved formulations for such compositions.

Accordingly, one aspect of the present invention is a mousse composition which can provide good cosmetic properties to keratinous materials upon which they have been applied, properties such as, for example, good transfer-resistance, and/or feel properties.

### SUMMARY OF THE INVENTION

The present invention relates to a mousse composition comprising water, at least one C2-C5 alcohol, at least one salt, at least one amphiphilic polymer, and at least one hydrophobic surface-treated pigment. Preferably, the mousse composition is free of, or substantially free of, propellants, waxes, oils, surfactants and/or film formers.

The present invention relates to methods for enhancing the appearance of a keratinous material comprising applying a mousse composition comprising water, at least one C2-C5 alcohol, at least one salt, at least one amphiphilic polymer, and at least one hydrophobic surface-treated pigment to a keratinous material in an amount sufficient to enhance the appearance of the keratinous material. Preferably, the mousse composition is free of, or substantially free of, propellants, waxes, oils, surfactants and/or film formers.

The present invention relates to methods for making-up a keratinous material comprising applying a mousse composition comprising water, at least one C2-C5 alcohol, at least one salt, at least one amphiphilic polymer, and at least one hydrophobic surface-treated pigment to a keratinous material in an amount sufficient to make-up the keratinous material. Preferably, the mousse composition is free of, or substantially free of, propellants, waxes, oils, surfactants and/or film formers.

The present invention relates to methods for making a mousse composition comprising combining a hydrophobic surface-treated pigment with water, at least one C2-C5 alcohol, at least one salt, and at least one amphiphilic polymer, and forming a mouse composition are provided. Preferably, the mousse composition is free of, or substantially free of, propellants, waxes, oils, surfactants and/or film formers.

It is to be understood that both the foregoing general description and the following detailed description are exemplary and explanatory only, and are not restrictive of the invention.

### DETAILED DESCRIPTION OF THE INVENTION

According to the invention, the term “free of” a particular type of material or individual material refers to a composition not containing the material in an amount sufficient to function in the desired manner. So, for example, a composition “free of” propellant does not contain a sufficient amount of propellant ingredient(s) to function as a propellant, although it may contain propellant ingredients in minor, non-functional amounts. Similarly, a composition “free of” surfactant does not contain a sufficient amount of surfactant ingredient(s) to function as a surfactant, although it may contain surfactant ingredients in minor, non-functional amount. Also, a composition “free of” film-former does not contain a sufficient amount of surfactant ingredient(s) to function as a film-former, although it may contain film former ingredients in minor, non-functional amount. Preferably, a composition “free of” a material contains 0.25% by weight of the composition or less of the material, preferably 0.20% by weight of the composition or less of the material, preferably 0.10% by weight of the composition or less of the material, and preferably 0% of the material. So, for example, compositions of the present invention which are “free of” wax and/or oils preferably contain 0.25% or less by weight of wax and/or oil, etc. “Substantially free of” means that 0.5% by weight of the composition or less of the identified material is present in the composition.

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

“Film former” or “film forming agent” or “film forming polymer” or “film forming resin” as used herein mean a polymer or resin that leaves a film on the substrate to which it is applied, for example, after a solvent accompanying the film former has evaporated, absorbed into and/or dissipated on the substrate.

“Transfer resistance” as used herein refers to the resistance of a color cosmetic composition from being removed from its site of application, in particular a keratinous material such as skin or lips. An example of a suitable testing protocol to determine if topcoat compositions have a positive or negative effect on the transfer-resistance of a color cosmetic composition is as follows: a 1 mil film of color cosmetic (e.g., foundation) is applied to scrub paper, and allowed to dry overnight in a 37° C. oven. The film is then placed on an automatic draw-down machine set-up with a draw-down rod that has loop Velcro strips attached to it. 5-6 trials can be run, with a standard included (e.g., a mousse composition devoid of powders). A small amount of sample is placed onto the film in front of a Velcro piece, and 6 passes across the film are made. After 6 passes have been made, the trials are rated vs. the standard using the following scale: 1-2 (removal worse); 3 (removal equal); 4-5 (removal better).

“Long wear” compositions as used herein, refers to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to human hair, skin or lips and evaluating the color of the composition after an extended period of time. For example, the color of a composition may be evaluated immediately following application to hair, skin or lips and these characteristics may then be re-evaluated and compared after a certain amount of time. Further, these characteristics may be evaluated with respect to other compositions, such as commercially available compositions. For lip compositions, “long wear” typically means the composition remains on the lips at least about 4 hours up to about 24 hours, and retains rich color even after eating.

“Substituted” as used herein, means comprising at least one substituent. Non-limiting examples of substituents include atoms, such as oxygen atoms and nitrogen atoms, as well as functional groups, such as amine groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Comprising” it is meant that other steps and/or ingredients which do not affect the end result may be added. The products, compositions, methods and processes of the present invention can include all the essential elements and limitations of the invention described herein as well as any of the additional or optional ingredients, components, steps, or limitations described herein.

As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3, 2-4, 1-4, etc.

As used herein a range of ratios is meant to include every specific ratio within, and combination of subranges between the given ranges.

“Keratinous materials” includes materials containing keratin such as hair, skin, eyebrows, lips and nails.

“Volatile”, as used herein, means having a flash point of less than about 100° C.

“Non-volatile”, as used herein, means having a flash point of greater than about 100° C.

“Gel Crossover Point” (Sol/Gel Point), means the point at which the G″ (loss modulus) intersects the G′ (storage modulus), reported in % strain. It is the point at which a composition goes from a more solid state to a more liquid state. An example of a method for determining gel crossover point is as follows: G″ (loss modulus) and G′ (storage modulus) using a Discovery HR-2 Rheometer by TA Instruments, having 40 mm parallel plate geometry on a stainless steel flat peltier plate. The test can be run @ 20° C., with test parameter of angular frequency of 1.0 rad/s and logarithmic sweep: Strain % 0.01 to 1000.0%. 5 points per decade. Results reported in % strain. Complex modulus (G*) is the magnitude (square root of the sum of the squares) of G′ and G″ as determined in the linear regime (at % strain less than the Gel Crossover Point).

“Mousse composition” as herein refers to a composition comprising a gas/air dispersion. Preferably, the mousse compositions of the present invention have a gel crossover point of >85% strain, preferably >94% strain, preferably 110% strain and preferably >150% strain, and preferably less than 500% strain, preferably less than 300% strain, and preferably less than 250% strain, including all ranges and subranges therebetween such as, for example, 94% strain to 250% strain, etc. Also preferably, the compositions of the present invention have a G* (complex modulus) of >100 Pa, preferably >1000 Pa, preferably >3000 Pa and preferably >5000 Pa, and preferably less than 50,000 Pa, preferably less than 35,000 Pa and preferably less than 10,000 Pa, including all ranges and subranges therebetween such as, for example, 1000 Pa to 35,000 Pa.

The compositions and methods of the present invention can comprise, consist of, or consist essentially of the essential elements and limitations of the invention described herein, as well as any additional or optional ingredients, components, or limitations described herein or otherwise useful.

Mousse Composition

According to the present invention, mousse compositions comprising water, at least one C2-C5 alcohol, at least one salt, at least one amphiphilic polymer, and at least one hydrophobic surface-treated pigment are provided. Preferably, the mousse composition is free of, or substantially free of, propellants, waxes, oils, surfactants and/or film formers.

C2-C5 Alcohol and Water (Hydro-Alcoholic Solvent Component)

According to the present invention, mousse compositions comprising at least one C2-C5 alcohol are provided. Suitable C2-C5 alcohols include ethanol, propanol, butanol, pentanol, isopropanol, isobutanol and isopentanol. Ethanol is particularly preferred.

Preferably, the C2-C5 alcohol(s) is/are present in the mousse compositions of the present invention in amounts ranging from about 1% to about 40%, preferably from about 3% to about 35%, and preferably from about 5% to about 30%, by weight, based on the total weight of the composition, including all ranges and subranges in between.

According to preferred embodiments, the mousse compositions of the present invention further comprise water. Preferably, the mousse compositions comprise from about 5% to about 90% water, preferably from about 20% to about 85% water, preferably from about 50% to about 85% water, and preferably from about 60% to about 80% water by weight, based on the total weight of the composition, including all ranges and subranges therebetween.

Preferably, the solvent component of the mousse compositions of the present invention consists essentially of, or consists of, water and C2-C5 alcohols. Preferably, the solvent component of the mousse composition is “free of” or “substantially free of” solvents other than water and C2-C5 alcohols.

According to preferred embodiments, the mousse compositions of the present invention are free of, or substantially free of, oils.

According to preferred embodiments, the mousse compositions of the present invention are free of, or substantially free of, surfactants.

According to preferred embodiments, the mousse compositions of the present invention are free of, or substantially free of, waxes.

According to preferred embodiments, the mousse compositions of the present invention are free of, or substantially free of, film formers.

According to preferred embodiments, the mousse compositions of the present invention are free of, or substantially free of, propellants.

According to preferred embodiments, the mousse compositions of the present invention are free of, or substantially free of, at least one of propellants, oils, surfactants, waxes and film formers; preferably at least two of propellants, oils, surfactants, waxes and film formers; preferably at least three of propellants, oils, surfactants, waxes and film formers; preferably at least four of propellants, oils, surfactants, waxes and film formers; and preferably of all five of propellants, oils, surfactants, waxes and film formers.

Amphiphilic Polymers

According to the present invention, mousse compositions comprising at least one amphiphilic polymer are provided. Preferably, the amphiphilic polymer comprises at least one ethylenically unsaturated monomer containing a sulphonic group, in freeform or partially or totally neutralized form and comprising at least one hydrophobic portion. The hydrophobic portion present in these polymers preferably contains from 6 to 50 carbon atoms, preferably from 6 to 22 carbon atoms, preferably from 6 to 18 carbon atoms and preferably from 12 to 18 carbon atoms, including all ranges and subranges therebetween.

The amphiphilic polymers may be based on at least one ethylenically unsaturated hydrophilic monomer A and on at least one hydrophobic monomer B. Preferably, the monomer A comprises a strong acid function, in particular a sulphonic acid or phosphonic acid function. The hydrophobic monomer B comprises at least one hydrophobic radical, chosen from: saturated or unsaturated C₆-C₁₈ linear alkyl radicals (for example, n-hexyl, n-octyl, n-decyl, n-hexadecyl, n-dodecyl or oleyl); branched alkyl radicals (for example, isostearic) or cyclic alkyl radicals (for example, cyclododecane or adamantane); C₆-C₁₈ fluoro or alkylfluoro radicals (for example, the group of formula —(CH₂)₂—(CF₂)₉—CF₃); a cholesteryl radical or radicals derived from cholesterol (for example, cholesteryl hexanoate); aromatic polycyclic groups, for instance naphthalene or pyrene; and silicone or alkylsilicone or alkylfluorosilicone radicals. Among these radicals, linear and branched alkyl radicals are preferred.

The amphiphilic polymers are preferably water-soluble or water-dispersible in neutralized form. Their viscosity at a concentration of 1% in water, at a shear rate of 1 s⁻¹, at a pH of between 5 and 8, at 25° C., is preferably less than 5 000 mPa·s. Amphiphilic polymers suitable for use in the present invention are described in US 2001/049419 and U.S. Pat. No. 6,645,476, the entire contents of both of which are hereby incorporated by reference.

The amphiphilic polymers may be crosslinked. The crosslinking agents may be chosen from, for example, the polyolefinically unsaturated compounds commonly used for crosslinking polymers obtained by free-radical polymerization. According to one preferred embodiment of the invention, the crosslinking agent is chosen from methylenebisacrylamide, allyl methacrylate or trimethylolpropane triacrylate (TMPTA). The degree of crosslinking preferably ranges from 0.01 mol % to 10 mol %, and preferably from 0.2 mol % to 2 mol %, relative to the polymer, including all ranges and subranges therebetween.

Preferred amphiphilic polymers are chosen from crosslinked or non-crosslinked amphiphilic polymers of 2acrylamido-2-methylpropanesulphonic (AMPS) acid and of at least one ethylenically unsaturated monomer comprising at least one hydrophobic portion containing from 6 to 30 carbon atoms, preferably from 6 to 22 carbon atoms, preferably from 6 to 18 carbon atoms and preferably from 12 to 18 carbon atoms, including all ranges and subranges therebetween.

The amphiphilic polymers can be partially or totally neutralized with a mineral base (for example, sodium hydroxide, potassium hydroxide or aqueous ammonia) or an organic base such as monoethanolamine, diethanolamine, triethanolamine, aminomethylpropanediol, N-methylglucamine, or basic amino acids, for instance arginine and lysine, and mixtures thereof.

Suitable examples of amphiphilic polymers include, but are not limited to, hydrophobically-modified sulfonic acid copolymers such as Ammonium Acryloyldimethyltaurate/VP Copolymer (Aristoflex AVC from Clariant), Ammonium Acryloyldimethyltaurate/Beheneth-25 Methacrylate Crosspolymer (Aristoflex HMB from Clariant) (crosslinked ethoxylated AMPS/behenyl methacrylate), Ammonium Acryloyldimethyltaurate/Steareth-25 Methacrylate Crosspolymer (Aristoflex HMS) (ethoxylated copolymer of AMPS/stearyl methacrylate crosslinked with trimethylol triacrylate), Aristoflex SNC (crosslinked ethoxylated AMPS/C16-C18), Aristoflex LNC (noncrosslinked AMPS/C12-C14), and mixtures thereof.

Preferably, the amphiphilic polymer(s) is/are present in the mousse compositions of the present invention in amounts ranging from about 0.05% to about 5% by weight, preferably from 0.075% to 2% by weight, preferably from 0.1% to 1% and preferably from 0.1% to 0.5% by weight, including all ranges and subranges therebetween, all weights based on the weight of the composition as a whole.

Salt

According to the present invention, mousse compositions comprising at least one salt are provided. The salt(s) of the invention may be anhydrous or hydrated. Suitable salts include, but are not limited to alkali metal salts, alkaline-earth metal salts and mixtures thereof. Alkali metals include lithium, sodium and potassium. Alkaline-earth metals include barium, calcium, magnesium, strontium and beryllium. However, the salt does not need to include an alkali metal or alkaline-earth metal, and can include other positively charged elements such as, for example, ammonium or zinc.

Salts may be, for example, carbonates, bicarbonates, sulphates, glycerophosphates, borates, chlorides, nitrates, acetates, hydroxides, persulphates, salts of alpha-hydroxy acids (for example, citrates, tartrates, lactates, malates), salts of fruit acids, salts of amino acids (for example, aspartate, arginate, glycoholate, fumarate, glutamate), and salts of fatty acids (for example, palmitate, oleate, caseinate, behenate). Preferably, the salt is a salt of (1) an alkali metal or alkaline earth metal and (2) nitrate, borate, chloride, sulphate, acetate, alpha hydroxy acid or amino acid. Specific examples of preferred salts include sodium chloride, magnesium chloride, and calcium chloride.

The at least one salt does not have to be present in the mouse compositions of the present invention as a separate compound or ingredient. Rather, the salt may be present as part of another compound in the composition. For example, the hydrophobic surface treatment of the hydrophobic surface-treated pigment may comprise at least one salt.

Preferably, the salt(s) is/are present in the mousse compositions of the present invention in amounts ranging from about 0.01% to about 15% by weight, preferably from 0.05% to 12.5% by weight, and preferably from 0.1% to 10% by weight, including all ranges and subranges therebetween, all weights based on the weight of the composition as a whole.

Hydrophobic Surface-Treated Pigment

According to the present invention, mousse compositions comprising at least one hydrophobic surface-treated pigment are provided. Examples of suitable pigments include, but are not limited to, inorganic pigments, organic pigments, lakes, pearlescent pigments, iridescent or optically variable pigments, and mixtures thereof. A pigment should be understood to mean inorganic or organic, white or colored particles.

In accordance with the present invention, such pigments are hydrophobically surface treated. For example, the pigments have totally or partially undergone a surface treatment chosen from chemical, electronic, electrochemical, mechanochemical, and mechanical surface treatments, with at least one organic agent which provides the pigment with a hydrophobic surface treatment. Suitable organic agents which can provide a hydrophobic surface treatment include, for example, sodium, potassium, magnesium, iron, titanium, zinc, and aluminum salts of amino acids, for example disodium stearoyl glutamate; waxes, for example carnauba wax and beeswax; fatty acids, fatty alcohols and derivatives thereof, such as stearic acid, hydroxystearic acid, stearyl alcohol, hydroxystearyl alcohol, and lauric acid and derivatives thereof; sodium, potassium, magnesium, iron, titanium, zinc, and aluminum salts of fatty acids, for example aluminum stearate and laurate; polyethylene; (meth)acrylic polymers, for example polymethyl methacrylates; polymers and copolymers comprising acrylate units; proteins; alkanolamines; silicone compounds comprising acrylate units; silicone compounds, for example silicones, polydimethylsiloxanes, alkoxysilanes, alkylsilanes, and siloxysilicates; organofluorine compounds, for example perfluoroalkyl ethers; and fluorosilicone compounds.

For example, the hydrophobic surface treatment of the pigments may be chosen from the following treatments: PEG-silicone treatments; triethoxycaprylylsilane treatments; methicone treatments; dimethicone treatments; dimethicone/trimethyl siloxysilicate treatments; lauroyllysine treatments; lauroyllysine dimethicone treatments; magnesium myristate treatments; aluminum dimyristate treatments; isostearyl sebacate treatments; disodium stearoyl glutamate treatments; dimethicone/disodium stearoyl glutamate treatments; acrylate/dimethicone copolymer and perfluoroalkyl phosphate treatments; polymethylhydrogenosiloxane/perfluoroalkyl phosphate treatments; lauroyllysine/aluminium tristearate treatments; octyltriethylsilane treatments; octyltriethylsilane/perfluoroalkyl phosphate treatments; acrylate/dimethicone copolymer treatments; and isopropyl titanium triisostearate treatments.

For the purposes of the present invention, the surface treatment is such that a surface-treated pigment conserves its intrinsic pretreatment pigmenting properties.

For purposes of the present invention, the hydrophobic surface-treated pigment may comprise the at least one salt.

The at least one surface-treated pigment may be present in the composition in total amounts generally ranging from 0.05% to 50% by weight, for example, from 0.1% to 35% by weight, or from 0.5% to 20% by weight relative to the total weight of the composition.

Preferably, the weight ratio of hydrophobic surface-treated pigment to amphiphilic polymer present in the mousse compositions of the present invention ranges from about 300:1 to about 1:3, preferably from about 200:1 to about 1:2, preferably from about 100:1 to about 1:1, and preferably from about 50:1 to about 5:1, including all ranges and subranges therebetween.

Additional Ingredients

According to preferred embodiments, the mousse compositions can comprise additional ingredients typically included in cosmetic compositions. A non-exhaustive discussion of such ingredients is set forth below.

Film-Forming Agents

According to preferred embodiments, the mousse compositions may optionally further comprise as least one film-forming agent. Preferably, if a film-forming agent is present in the mousse composition, the film-forming agent is a silicone film-forming agent selected from the group consisting of silicone resins, polyorganosiloxane copolymers, and mixtures thereof. Also preferably, the silicone resin is selected from the group consisting of siloxysilicate resins, silsesquioxane resins, and mixtures thereof.

Volatile Oil

The compositions of the invention may optionally further comprise at least one volatile oil.

Examples of suitable volatile solvents include volatile hydrocarbon-based oils such as, for example, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, isohexadecane, and for example, the oils sold under the trade names of Isopar or Permethyl, the C₈ to C₁₆ branched esters such as isohexyl or isodecyl neopentanoate, alcohols, and their mixtures. Preferably, the volatile hydrocarbon-based oils have a flash point of at least 40° C.

Examples of volatile hydrocarbon-based oils include, but are not limited to those given in Table 1 below.

TABLE 1

| Compound | Flash Point (° C.) |
|---|---|
| Isododecane | 43 |
| Isohexadecane | 102 |
| Isodecyl neopentanoate | 118 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

The volatile solvent may also be chosen from volatile silicone oils, which may be linear or cyclic, having a viscosity, at room temperature, of less than or equal to 6 cSt, and having from 2 to 7 silicon atoms, optionally substituted with alkyl or alkoxy groups of 1 to 10 carbon atoms.

Examples of suitable volatile silicone oils include, but are not limited to, those listed in Table 2 below.

TABLE 2

| | | Viscosity |
|---|---|---|
| Compound | Flash Point (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or | | |
| D4) | | |
| Dodecamethylcyclohexasiloxane | 93 | 7 |
| (D6) | | |
| Decamethyltetrasiloxane(L4) | 63 | 1.7 |
| KF-96 A from Shin Etsu | 94 | 6 |
| PDMS (polydimethylsiloxane) | 56 | 1.5 |
| DC 200 (1.5 cSt) from Dow | | |
| Corning | | |
| PDMS DC 200 (2 cSt) from Dow | 87 | 2 |
| Corning | | |
| PDMS DC 200 (5 cSt) from Dow | 134 | 5 |
| Corning | | |
| PDMS DC 200 (3 St) from Dow | 102 | 3 |
| Corning | | |

The at least one volatile solvent, if present, is generally present in the compositions of the present invention in an amount ranging from about 5% to about 50% by weight; such as from about 10% to about 45% by weight; such as from about 15% to about 40% by weight, all weights being based on the weight of the composition as a whole.

Non-Volatile Oil

The compositions of the present invention may optionally further comprise at least one non-volatile oil.

The volatility of the oils can be determined using the evaporation speed as set forth in U.S. Pat. No. 6,338,839, the content of which is herein incorporated by reference.

Non-volatile oils include low viscosity oils (having a viscosity from about 5 to about 10 centipoise) and high viscosity oils (having a viscosity of from about 100 to about 10,000 centipoise), and mixtures thereof.

According to a particular embodiment of the present invention, the oil is a high viscosity oil which is a silicone oil and/or a hydrocarbon oil. “High viscosity” means an oil having a viscosity greater than 100 cSt, particularly greater than 250 cSt at 25° C. Most particularly, the non-volatile oil is selected from a silicone oil. Such oils are described, for example in US 2011/0293550 and US 2004/0126350, both of which are herein incorporated by reference.

Non-limiting examples of suitable non-volatile silicone oils include polymethylsiloxanes with a linear or cyclic silicone chain, which are liquid or pasty at room temperature, especially cyclopolydimethylsiloxanes (cyclomethicones) such as cyclohexasiloxane; polydimethyl-siloxanes (CTFA designation “dimethicones”) comprising alkyl or alkoxy groups, which are pendent or at the end of a silicone chain, these groups containing from 2 to 24 carbon atoms; polydiethyl siloxanes; and dimethicone fluids having viscosity from about 300 cPs at 25° C. to about 1500 cPs at 25° C. Particularly useful dimethicone fluids have viscosity from about 350 cPs at 25° C. to about 1000 cPs at 25° C.

Specific examples of suitable for this invention high viscosity silicone oils include, but are not limited to, Xiameter® silicone fluids from Dow Corning.

Wax

The mousse compositions of the present invention may optionally further comprise at least one wax.

Additional Coloring Agent (Colorant)

The compositions of the present invention may optionally further comprise at least one cosmetically acceptable colorant in addition to the hydrophobic-surface treated pigment such as another type of pigment or dyestuff. Examples of suitable pigments include, but are not limited to, inorganic pigments, organic pigments, lakes, pearlescent pigments, iridescent or optically variable pigments, and mixtures thereof. A pigment should be understood to mean inorganic or organic, white or colored particles.

Representative examples of inorganic pigments useful in the present invention include those selected from the group consisting of rutile or anatase titanium dioxide, coded in the Color Index under the reference CI 77,891; black, yellow, red and brown iron oxides, coded under references CI 77,499, 77, 492 and, 77,491; manganese violet (CI 77,742); ultramarine blue (CI 77,007); chromium oxide (CI 77,288); chromium hydrate (CI 77,289); and ferric blue (CI 77,510) and mixtures thereof.

Representative examples of organic pigments and lakes useful in the present invention include, but are not limited to, D&C Red No. 19 (CI 45,170), D&C Red No. 9 (CI 15,585), D&C Red No. 21 (CI 45,380), D&C Orange No. 4 (CI 15,510), D&C Orange No. 5 (CI 45,370), D&C Red No. 27 (CI 45,410), D&C Red No. 13 (CI 15,630), D&C Red No. 7 (CI 15,850), D&C Red No. 6 (CI 15,850), D&C Yellow No. 5 (CI 19,140), D&C Red No. 36 (CI 12,085), D&C Orange No. 10 (CI 45,425), D&C Yellow No. 6 (CI 15,985), D&C Red No. 30 (CI 73,360), D&C Red No. 3 (CI 45,430) and the dye or lakes based on cochineal carmine (CI 75,570) and mixtures thereof.

Representative examples of pearlescent pigments useful in the present invention include those selected from the group consisting of the white pearlescent pigments such as mica coated with titanium oxide, mica coated with titanium dioxide, bismuth oxychloride, titanium oxychloride, colored pearlescent pigments such as titanium mica with iron oxides, titanium mica with ferric blue, chromium oxide and the like, titanium mica with an organic pigment of the above-mentioned type as well as those based on bismuth oxychloride and mixtures thereof

Miscellaneous Additives

The compositions of the present invention may optionally further comprise at least one cosmetically or dermatologically acceptable additive such as thickener, a plasticizer, an antioxidant, an essential oil, a botanical extract, a preserving agent, a fragrance, a pasty fatty substance, a neutralizing agent, and cosmetically active agents and/or dermatological active agents such as, for example, emollients, moisturizers, vitamins, essential fatty acids and medicaments.

The compositions of the present invention can be stored in any container suitable for storing mousse compositions. The container can be made of any materials known in the art as suitable for containing mousse compositions such as, for example, metal, glass, plastic, etc. Suitable containers may also optionally include a dispensing element such as a nozzle for providing mousse composition from the container to the user.

According to preferred embodiments, methods for enhancing the appearance of a keratinous material comprising applying a mousse composition comprising water, at least one C2-C5 alcohol, at least one salt, at least one amphiphilic polymer, and at least one hydrophobic surface-treated pigment to a keratinous material in an amount sufficient to enhance the appearance of the keratinous material are provided. Preferably, the mousse composition is free of, or substantially free of, propellants, waxes, oils, surfactants and/or film formers.

According to preferred embodiments, for methods for making-up a keratinous material comprising applying a mousse composition comprising water, at least one C2-C5 alcohol, at least one salt, at least one amphiphilic polymer, and at least one hydrophobic surface-treated pigment to a keratinous material in an amount sufficient to make-up the keratinous material are provided. Preferably, the mousse composition is free of, or substantially free of, propellants, waxes, oils, surfactants and/or film formers.

The compositions and methods of the present invention can “comprise,” “consist of” or “consist essentially of” the identified ingredients and process steps. For purposes of the compositions and methods of the present invention where the invention “consists essentially of” the identified ingredients and/or process steps, the sole “basic and novel property” of such compositions and/or methods is formation of a mousse composition.

According to preferred embodiments, methods of making mousse compositions comprising combining a hydrophobic surface-treated pigment with water, at least one C2-C5 alcohol, at least one salt, and at least one amphiphilic polymer, and forming a mouse composition are provided. Preferably, the mousse composition is free of, or substantially free of, propellants, waxes, oils, surfactants and/or film formers.

According to preferred embodiments, methods of making a mousse composition comprising (1) mixing an amphiphilic polymer with water to form a mixture, allowing the amphiphilic polymer to swell in the mixture, (2) adding C2-C5 alcohol to the mixture to form a C2-C5 alcohol-containing mixture, (3) optionally adding salt to the C2-C5 alcohol-containing mixture under mixing at a shear rate above 2,500 RPM, preferably between about 3,000 RPM and 7,000 RPM, and preferably from about 4,000 RPM to about 6,000 RPM with 5,000 RPM being most preferred for greater than 5 minutes, preferably from about 5 minutes to 60 minutes, preferably from about 15 minutes to 50 minutes, and preferably from about 20 minutes to 40 minutes, including all ranges and subranges therebetween to form a salt-containing mixture, and (4) adding hydrophobic surface-treated pigments to either the C2-C5 alcohol-containing mixture from (2) or the salt-containing mixture from (3) under mixing at shear rates and durations discussed above. If the hydrophobic surface-treated pigment comprises at least one salt, it is not necessary to add salt to the C2-C5 alcohol-containing mixture, making step (3) optional. However, if the hydrophobic surface-treated pigment does not comprise at least one salt, salt should be added to the C2-C5 alcohol-containing mixture. After allowing the mixture to rest, a mouse composition is formed.

According to preferred embodiments, mousse compositions of the present invention comprise:

from about 5% to about 30% by weight of ethanol;

from about 60% to about 80% by weight of water;

from about 0.1% to about 0.5% by weight of Acryloyldimethyltaurate/Steareth-25 Methacrylate Crosspolymer;

from about 0.1% to about 10% by weight of at least one salt; and

from about 0.5% to about 20% by weight of at least one hydrophobic surface-treated pigment.

According to preferred embodiments, mousse compositions of the present invention comprise:

from about 5% to about 30% by weight of ethanol;

from about 60% to about 80% by weight of water;

from about 0.1% to about 0.5% by weight of Acryloyldimethyltaurate/Steareth-25 Methacrylate Crosspolymer;

from about 0.1% to about 10% by weight of at least one salt selected from the group consisting of calcium chloride, magnesium chloride and sodium chloride, and mixtures thereof; and

from about 0.5% to about 20% by weight of at least one hydrophobic surface-treated pigment.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

The present invention will be better understood from the examples which follow. The examples are intended to be nonrestrictive and explanatory only, with the scope of the invention defined by the claims.

### Example 1—Comparative Compositions Containing Hydrophilic Surface Coated Pigment

The following composition can be prepared (with at least one of the identified pigments present):

| | INVENTION |
|---|---|
| INCI US | COMPOSITION |
| WATER | 5-90 |
| AMMONIUM | 0.1-0.5 |
| ACRYLOYLDIMETHYLTAURATE/STEARETH-25 | |
| METHACRYLATE CROSSPOLYMER | |
| ETHANOL | 5-30 |
| SALT (NaCl) | 0.01-10 |
| TITANIUM OXIDE (and) PHYTIC ACID | 0-25 |
| IRON OXIDE (and) PHYTIC ACID - YELLOW | 0-25 |
| IRON OXIDE (and) PHYTIC ACID - RED | 0-25 |
| IRON OXIDE (and) PHYTIC ACID - BLACK | 0-25 |

### Example 2—Invention Compositions Containing Hydrophobic Surface Treated Pigments

The following composition can be prepared (with at least one of the identified pigments present):

| | INVENTION |
|---|---|
| INCI US | COMPOSITION |
| WATER | 5-90 |
| AMMONIUM | 0.1-0.5 |
| ACRYLOYLDIMETHYLTAURATE/STEARETH-25 | |
| METHACRYLATE CROSSPOLYMER | |
| ETHANOL | 5-30 |
| SALT (NaCl) | 0.01-10 |
| TITANIUM OXIDE (and) ALUMINA (and) | 0-25 |
| ISOPROPYL TITANIUM TRIISOSTEARATE | |
| IRON OXIDE (and) ISOPROPYL TITANIUM | 0-25 |
| TRIISOSTEARATE - YELLOW | |
| IRON OXIDE (and) ISOPROPYL TITANIUM | 0-25 |
| TRIISOSTEARATE - RED | |
| IRON OXIDE (and) ISOPROPYL TITANIUM | 0-25 |
| TRIISOSTEARATE - BLACK | |

### Example 3—Comparison of Compositions Containing Different Types of Surfactants

Compositions containing amphiphilic polymer, water, salt, ethanol and pigment according to examples 1 and 2 were prepared. The amphiphilic polymer was allowed to hydrate and swell in water during mixing. Ethanol was added to the system. Salt was then added and mixed at 5,000 RPM for 5 minutes. The pigments were added to the system and mixed at 5,000 RPM for 30 min. The system was allowed to sit overnight. Interestingly, only the hydrophobic surface treated pigments formed a mousse texture upon the addition of salt.

| | MOUSSE TEXTURE | MOUSSE TEXTURE |
|---|---|---|
| | WITHOUT SALT | WITH ADDED SALT |
| | (YES - Invention | (YES - Invention |
| | Composition; | Composition; |
| | NO - Comparative | NO - Comparative |
| SURFACE TREATMENT | Composition) | Composition) |
| ALGINATE | NO | NO |
| PHYTIC ACID | NO | NO |
| ISOPROPYL TITANIUM | NO | YES |
| TRIISOSTEARATE | | |
| TRIETHOXYCAPRYLYLSILANE | NO | YES |
| PERFLUOROOCTYL | NO | YES |
| TRIETHOXYSILANE | | |
| METHICONE | NO | YES |
| DISODIUM STEAROYL | YES | YES |
| GLUTAMATE (and) ALUMINUM | | |
| HYDROXIDE | | |

### Example 4—Evaluation of Rheological Properties

The rheological properties of the compositions of example 3 were evaluated to compare the systems in the absence and presence of a salt. G* (complex modulus) provides insight on the flexibility and stiffness of a material. The cross-over point indicates the amount of shear that is required to collapse the microstructure (G′>G′) and allow the material to flow. The increase in both the cross-over point and G* upon the addition of salt indicated the formation of a mousse texture.

| | NO SALT | | ADDITION OF SALT | |
|---|---|---|---|---|
| | CROSS OVER | | CROSS OVER | G* |
| SURFACE TREATMENT | POINT (%) | G* (Pa) | POINT (%) | (Pa) |
| ALGINATE | 12.3 | 0.53 | 5.0 | 0.32 |
| PHYTIC ACID | 31.6 | 0.63 | 12.6 | 5.5 |
| ISOPROPYL TITANIUM | 13.2 | 23.8 | 187.2 | 7911.8 |
| TRIISOSTEARATE | | | | |
| TRIETHOXYCAPRYLYLSILANE | 11.5 | 10.9 | 94.9 | 30,364.60 |
| PERFLUOROOCTYL | 5.5 | 7.2 | 117.1 | 9378.6 |
| TRIETHOXYSILANE | | | | |
| METHICONE | 8.9 | 6.8 | 222.4 | 1404.5 |
| DISODIUM STEAROYL | 94.1 | 462.4 | 175.2 | 4219.4 |
| GLUTAMATE (and) ALUMINUM | | | | |
| HYDROXIDE | | | | |

1. A mousse composition comprising water, at least one C2-C5 alcohol, at least one salt, at least one amphiphilic polymer, and at least one hydrophobic surface-treated pigment, wherein the mousse composition is free of propellants and wherein the at least one hydrophobic surface-treated pigment has a hydrophobic surface treatment comprising at least one of salt(s) of amino acid(s), salt(s) of fatty acid(s), silicone compound(s), organofluorine compound(s), and fluorosilicone compound(s).

2. The mousse composition of claim 1, wherein the mousse composition is free of waxes and film formers.

3. The mousse composition of claim 1, wherein the mousse composition is free of oils.

4. The mousse composition of claim 1, wherein the mousse composition is free of surfactants.

5. The mousse composition of claim 1, wherein the at least one C2-C5 alcohol is present in an amount of from about 5% to about 30% by weight of the total weight of the composition.

6. The mousse composition of claim 1, wherein the at least one C2-C5 alcohol is ethanol.

7. The mousse composition of claim 1, wherein the at least one salt is present in an amount of from about 0.1% to about 10% by weight of the total weight of the composition.

8. The mousse composition of claim 1, wherein the at least one salt is selected from the group consisting of calcium chloride, magnesium chloride, sodium chloride, and mixtures thereof.

9. The mousse composition according to claim 1, wherein the at least one amphiphilic polymer is selected from the group consisting of Ammonium Acryloyldimethyltaurate/VP Copolymer, Ammonium Acryloyldimethyltaurate/Beheneth-25 Methacrylate Crosspolymer, Ammonium Acryloyldimethyltaurate/Steareth-25 Methacrylate Crosspolymer, crosslinked ethoxylated AMPS/C16-C18, noncrosslinked AMPS/C12-C14, and mixtures thereof.

10. The mousse composition according to claim 1, wherein the at least one amphiphilic polymer is selected from the group consisting of Ammonium Acryloyldimethyltaurate/Beheneth-25 Methacrylate Crosspolymer, Ammonium Acryloyldimethyltaurate/Steareth-25 Methacrylate Crosspolymer, and mixtures thereof.

11. The mousse composition according to claim 1, wherein the at least one amphiphilic polymer is present in an amount of from about 0.1% to about 0.5% by weight of the total weight of the composition.

12. The mousse composition according to claim 1, wherein the at least one hydrophobic surface-treated pigment is present in an amount of from about 0.5% to about 20% by weight of the total weight of the composition.

13. The mousse composition according to claim 1, wherein the hydrophobic surface-treated pigment comprises the at least one salt.

14. The mousse composition according to claim 1, wherein the weight ratio of hydrophobic surface-treated pigment to amphiphilic polymer present ranges from about 50:1 to about 5:1.

15. A method of enhancing the appearance of a keratinous material comprising applying the mousse composition of claim 1 to the keratinous material in an amount sufficient to enhance the appearance of the keratinous material.

16. A method of making-up the appearance of a keratinous material comprising applying the mousse composition of claim 1 to the keratinous material in an amount sufficient to make-up the appearance of the keratinous material.

17. The mousse composition according to claim 1, wherein the G* (complex modulus) of the composition is greater than 1000 Pa.

18. The mousse composition according to claim 17, wherein the G* (complex modulus) of the composition is from 1000 Pa to 50,000 Pa.

19. The mousse composition according to claim 17, wherein the G* (complex modulus) of the composition is from 1000 Pa to 35,000 Pa.

20. The mousse composition according to claim 7, wherein the at least one hydrophobic surface-treated pigment is present in an amount of from about 0.5% to about 20% by weight of the total weight of the composition.

21. The mousse composition according to claim 1, wherein the at least one hydrophobic surface-treated pigment has a hydrophobic surface treatment comprising at least one of isopropyl titanium triisostearate, triethoxycaprylylsilane, perfluorooctyl triethoxysilane, methicone, and disodium stearoyl glutamate.
